(19) United States
(12) Patent Application Publication
(10) Pub. No.: US 2025/0026783 A1
(43) Pub. Date: Jan. 23, 2025

(54) Intermittent Percolation Washing

(71) Applicant: PolyPeptide Laboratories Holding (PPL) AB, Limhamn (SE)

(72) Inventors: Olivier Ludemann-Hombourger, Rohrwiller (FR); Renan Ravetti Duran, Strasbourg (FR)

(21) Appl. No.: 18/701,655

(22) PCT Filed: Oct. 16, 2022
(86) PCT No.: PCT/SE2022/050936

§ 371 Date: Apr. 16, 2024

(30) Foreign Application Priority Data
Oct. 18, 2021 (SE) 2130279-9

(51) Int. Cl. C07K 1/04; C07K 1/14

(52) U.S. Cl. CPC C07K 1/045; C07K 1/145

(57) ABSTRACT

The present invention relates to a process for the displacement of compounds and solvents comprised in the liquid and solid phase of a heterogeneous liquid-solid phase reaction, such as a solid phase peptide synthesis (SPPS) or solid phase oligonucleotide synthesis (SPOS). The process is applied in a reactor comprising a liquid phase and a solid phase, to which reactor a displacement liquid is fed discontinuously while the liquid phase is removed from the reactor chamber. The invention also encompasses a process for synthesizing peptides or oligonucleotides by the application of a solid phase peptide or oligonucleotide synthesis protocol further comprising the process for the displacement of compounds.

### FIELD OF THE INVENTION

The present invention relates to a process for the displacement of compounds and solvents comprised in the liquid and solid phase of a heterogeneous liquid-solid phase reaction, such as a solid phase peptide synthesis (SPPS) or solid phase oligonucleotide synthesis (SPOS). The process is applied in a reactor comprising a liquid phase and a solid phase, to which reactor a displacement liquid is fed discontinuously while the liquid phase is removed from the reactor chamber. The invention also encompasses a process for synthesizing peptides or oligonucleotides by the application of a solid phase peptide or oligonucleotide synthesis protocol further comprising the process for the displacement of compounds

### BACKGROUND OF THE INVENTION

Heterogeneous liquid-solid phase chemical reaction protocols are attractive for the synthesis of molecules comprising recurrent sub-units. The liquid-solid phase protocol enables the effective separation of the solid phase from the liquid phase providing suitable conditions for the application of recurrent cycles comprising reaction steps for the successive, step-wise introduction (addition) of sub-units. Liquid-solid phase reaction protocols have been successfully implemented in the field of peptide synthesis and oligonucleotide synthesis. Under the course of the synthesis the nascent molecule (such as a growing peptide or oligonucleotide) is covalently bound to the solid support providing the conditions for the efficient removal of by-products by washing between reaction steps of the recurrent cycles. While heterogeneous liquid-solid phase chemical reaction protocols have had a profound impact on the synthesis of e.g. peptides and oligonucleotides at commercial scales, the methodology consumes significant volumes of solvents needed for the displacement of reagents, by-products and reaction solutions after every reaction step of the recurrent cycles.

Solid phase peptide synthesis is usually performed in a batch reactor such as in a stirred-tank reactor (STR) where there is no continuous flow from or to the reactor during the reaction. Tubular reactors, such as packed-bed reactors, offer some advantages over other reactors during wash step. Packed bed reactors allow washing to proceed as a displacement operation, rather than a dilution operation as in an STR, provided the amount of “dead volume” between the reactor inlet and the resin bead is minimized.

Solid phase peptide synthesis allows peptide chains to be built on a solid support by a recurrent cycle comprising the steps: attaching an amino acid to the support, deprotecting the amino acid, and coupling one or more subsequent amino acids to the amino acid or amino acid fragment covalently bound to the solid support. The solid support used in solid-phase peptide synthesis is usually a gel resin with a low degree of cross-linking. The most common support is the polystyrene containing 1 or 2% or divinylbenzene (DVB) as a cross linking agent, but other solid support includes polyacrylate, polyacrylamide, and polyethylene glycol. These cross-linked supports are insoluble in organic solvents, but they are solvated and swell in aprotic solvents, such as toluene, dimethylformamide and dichloromethane. Moreover, the peptide resin may also sometimes shrink/swell during the assembly due to the growth of the peptide chain length. In addition, these resins tend to be fairly soft in nature and, thus, are sensitive to physical attrition.

During the peptide synthesis steps (coupling and deprotection), the preferred reactor is a stirred reactor, which allows to have a homogeneous medium for the control of the reaction. On the other hand, for the washing of the resin between the synthesis steps of a cycle, the preferred reactor is a column-type piston reactor, which allows the species to be removed to be percolated in an optimal way with a minimum of solvent.

Solid phase oligonucleotide synthesis has many similarities with solid phase peptide synthesis. The target nucleotide is formed by the consecutive reaction (coupling) of individual nucleosides on a solid phase by the application of recurrent reaction steps making up a cycle. More specifically, oligonucleotides are typically formed by the implementation of derivatives of nucleosides comprising protection groups, notably phosphoramidite. The key feature of phosphoamidates is the reactivity towards nucleophiles (e.g. the deprotected hydroxyl group of the 5′ carbon of the pentose sugare) catalyzed by weak acids such as tetrazole. The tetrazole catalyzed phosphoramidite coupling increases the efficiency to above 99% allowing the synthesis of long oligonucleotides, oligonucleotides of up to 100 nucleotides and above. Normally, the oligonucleotide is synthesized from the 3′ end to the 5′ end and starting with a suitable solid phase to which a protected nucleoside is covalently bound by way of a linker/spacer at the 3′ carbon. The reactive groups of the nucleoside, i.e. the hydroxyl groups, phosphate group and amine of the base, are typically protected. Suitably, the 5′ hydroxyl group is protected by dimethoxytrityl (DMT), isobutyryl or benzoyl is used for protecting the amine of the base and phosphoramidite protects the hydroxyl groups of the 3′ carbon. The phosphoramidite solid phase synthesis begins with the 3′ nucleotide and proceeds through a series of cycles composed of four reaction steps which are repeated until the ultimate 5′ nucleotide of the target oligonucleotide is attached. The four reaction steps of a phosphoramidite solid phase synthesis typically includes deprotection, coupling, capping and stabilization. The solid phase for oligonucleotide synthesis of choice has been and still is porous silicates such as controlled pore glass (CPG). However, recently also solid phases based on polystyrene has gained interest (e.g. porous cross-linked aminoethyl polystyrene resins). The oligonucleotide synthesis is usually implemented in a reaction column comprising a solid support. The reagents are flowed through the column sequentially.

As with solid phase peptide synthesis also solid phase oligonucleotide synthesis consumes significant amounts of liquids used to displace excess reactants, by-products and reaction solvents after the reaction steps of a cycle. The reduction of displacement liquid (washing liquid) is warranted since the displacement liquid represents half of the organic materials used equating to about 25% of the Process Mass Intensity (PMI=quantity of raw materials divided by quantity of API [Active Pharmaceutical Substance]).

Continuous flow reactors are usually deployed for solid phase oligonucleotide synthesis especially of the solid phase is porous silicate. One type of continuous flow reactor is the fixed bed reactor. As alluded to above the solid phase, usually a polymer with some cross-linking, tends to change volume specifically due to the solvent but also due to the growing peptide or oligonucleotide. It is not unusual that resins for SPPS swell by more than 100% volume-wise but also contract more than 100%. Somehow the swelling and contraction of the resin must be accommodated for in a continuous flow reactor.

U.S. Pat. No. 9,169,287 B1 discloses a continuous flow reactor for the solid phase peptide synthesis by the application of a packed bed reactor. For accommodating the volume change of the resin only a part of the reactor is filled with the resin while a significant volume of liquid is present above the resin bed. The extensive volume of liquid above the resin bed is highly unfavorable for the reactions steps such as coupling reaction and the performance of the washing due in part to back-mixing effects and dilution, resulting in the use of a large excess of reagents and a high consumption of washing liquid. The synthesis of U.S. Pat. No. 9,169,287 B1 would not be favorable for a large-scale production of peptides.

US 200206714 A1 presents a continuous flow reactor for the solid phase peptide synthesis. The solution for the accommodation of a resin with significant change of volume over reactions cycles while keeping the volume of the liquid phase low is the provision of modulating the volume of the entire reactor by a moveable wall actuated by a piston. The proposition of US 200206714 A1 presents a reactor of high complexity which may be difficult to operate favorable for a large-scale production.

Moreover, the use of flow reactors, in general, does not provide homogeneous reaction conditions during the reaction steps resulting of locally varying concentrations of reagent in the resin which may lead to variabilities of the reaction progress over the bed thereby impacting purity.

Furthermore, continuous flow reactors have a height much greater than its diameter, with a significant amount of wall surface area which impedes the expansion and contraction of the resin bed. When the resin bed is expanded, it packs against the walls of the vessel and reduces the void space between the resin particles. A high-pressure drop is thus generated to push all the wash through the bed in the required amount of time. This high pressure can compress the soft gel beads and potentially limit the flow, damage the resin beads, create fine particles which may block the filter, and potentially damage or break the filter frit. If high pressure is not used, then flow through the bed will be too slow and the washing step will take more time leading to a lower process productivity.

DE 2017351A1 discloses a reactor based upon the design of a rotating bowl have also been developed. Rotating bowls or centrifugal reactors can allow for increasing the liquid velocity relative to the resin particle. This “washing machine” reactor in which a porous basket is initially loaded with resin, then spun to a moderate speed while submerged in a liquid. The centrifugal forces cause the resin particles to form a bed on the inside walls of the basket and cause a moderate degree of fluid recirculation through the resin bed. The bowl is flooded (i.e. filled with liquid), as is the resin bed. The drag imposed upon the basket by the liquid bath imposes high torque upon the drive motor and will also cause the generation of heat. For these reasons, the rotational speed of the Birr reactor will be relatively slow and the relative fluid to solid velocity will be limited. The limits on rotational speed will almost certainly result in a non-uniform resin bed that is shallow at the top and deep near the bottom. The technology is also complex to scale-up for large scale manufacturing.

U.S. Pat. No. 5,186,824A discloses a centrifugal reactor based upon a flooded “hollow rotor” is disclosed. The liquid flow path in the reactor is axial rather than radial, and the geometry is irregular for the liquid flow fields. The point at which liquid is introduced depends upon the density of the liquid in relation to the density of the most recently added liquid. Also, little room is provided for expansion of the resin. As a result of expansion and contraction, the exposure of resin to the liquid phase is likely to be non-uniform. Complete and uniform contact and removal of liquid from the rotor may be very difficult to accomplish.

WO 2021/158444A2 relates to a reactor design for solid phase peptide synthesis. The hallmark of the reactor design is the application of several reactors in series. A serial reactor design has the potential of reducing solvents, however, at the cost of needing multiple reactors of high complexity which may be difficult to operate for a large-scale production.

US 2021/0094982 A1 discloses a reactor system for a solid phase peptide synthesis. The system may use percolation for the removal of solvents. Percolation is implemented in the reactor by introducing the washing liquid at the same flow rate as the outlet flowrate, thereby maintaining the height of the liquid at a constant level. It is stated that maintaining the height of the liquid constant during percolation by keeping the inflow and outflow at the same flowrate provides the most effective washing mode enabling a significant reduction of consumption of solvent.

A continuous flow of washing liquid makes it difficult to monitor the level of the liquid phase above the resin bed. By the application of a discontinuous feed of washing (displacement) liquid to the reactor while at the same time continuously remove the liquid phase from the reactor the liquid level (that is the height of the liquid phase above the solid phase (resin bed)) can be accurately monitored during the period where no washing liquid is fed to the reactor while simultaneously not significantly influencing the superficial velocity of the washing liquid over the solid phase (resin bed).

US 2021/0094982 A1 presents experimental data related to a percolation washing protocol with the inflow of washing liquid and outflow of liquid set at same flow rate. The table in par. 0144 presents different flow rates of washing solvent (and by inference also the flowrate of the outflow of liquid from the reactor) and the impact on the volume of washing liquid and time needed to reach a set endpoint (here: amount of piperidine in the liquid phase exiting the reactor). In US 2021/0094982 A1 the optimum washing is defined as a preferred endpoint. At a flow rate of 50 ml/min optimum washing is reached within 20 minutes at a consumption of 930 ml washing solvent. At a flow rate of 300 ml/min the optimum is reached within 4 minutes at a consumption of 1200 ml washing solvent. Obviously, both washing time and washing liquid consumed is of importance. At a set endpoint a decrease in washing time is offset by the increase in solvent consumption while a decrease in solvent consumption is offset by an increased washing time and reduced overall throughput.

One conclusion from the empirical data underlying the present invention is the finding that the metric provided by the superficial velocity is a more adequate proxy for the overall efficiency than the washing solvent flow rate. The superficial velocity, unlike the flow rate, is independent with respect to reactor size (universally applicable to reactors of different volumes) taking account to both washing solvent consumption and washing time. Moreover, the applicant has also surprisingly found that there is a specifically preferred range constituting an optimum of the superficial velocity. At a specific range of the superficial velocity the overall efficiency taking account to both spent volume of washing liquid and time is specifically advantageous.

The percolation washing of US 2021/0094982 A1 applies a constant liquid level as close as possible to the resin bed without generating disturbances. To provide a constant liquid level, the inflow and outflow of liquid must be continuous and having identical flowrates at steady state. The provision of a constant liquid level close to the resin bed is difficult to attain without sophisticated regulation system incorporating sensors. The volume of the resin bed may change as a function of the solvent (solvent composition) and the growth of the peptide chain length. Moreover, properties of the solid phase alter as the compound of interest covalently bound to the solid phase increases in size by the introduction of sub-units. When a washing liquid (displacement liquid) is introduced after a reaction step of a recurrent cycle the composition of the liquid phase gradually changes which may affect the swelling characteristics of the solid phase. In conclusion, not only the liquid level may need to be monitored but also the level of the solid phase (resin bed) for the reasons presented. Thus, to keep the liquid level constant as close as possible to the solid phase depends on the accurate monitoring of the level of the solid phase (i.e. the liquid-solid phase boundary) and the liquid level (i.e. gas-liquid phase boundary). In US 2021/0094982 the liquid level is measured during a continuous inflow of washing liquid, which impairs precision of the measurements.

The present invention implements a discontinuous inflow of the displacement liquid enabling the monitoring of the solid phase level and liquid level without the disturbance of a continuous feed of a washing liquid. Furthermore, the flowrate and duration of the discontinuous feed of displacement liquid can be easily modulated (without affecting the superficial velocity of the displacement liquid over the resin bed [solid phase]) if necessary for e.g. cleaning the walls of the reactor from solid phase (resin).

Furthermore, displacement of reaction solvents, reagents and by-products by the application of a discontinuous feed of displacement/washing liquid and the continuous purge of liquid phase form the reactor further improves the reduction of spent washing liquid over a washing protocol of a continuous inflow of washing liquid and continuous outflow of liquid.

### SUMMARY OF THE INVENTION

The present invention relates to a process for displacing compounds (and/or solvents) comprised in the liquid and solid phase of reaction steps of a heterogeneous chemical reaction, the solid phase being present in particulate form dispersed in the liquid phase, for the formation of a target-product which is synthesized by the consecutive introduction of sub-units (compounds) by repetitive (recurrent) cycles, each cycle comprising reaction steps, the process comprising providing at least means preventing the solid phase from escaping from the reaction chamber when solid phase is removed from the reactor; removing the liquid phase from the reactor; while a displacement liquid is discontinuously feed to the reactor, and wherein a permanent layer of liquid phase is maintained over the solid phase.

The invention also encompasses a process for the synthesis of peptides and oligonucleotides by the application of a solid synthesis protocol comprising the process for displacing compounds as presented herein.

Peptides and oligonucleotides are biological polymers comprising recurrent sub-units (building blocks) which are similar in many dimensions such as chemical structure. These sub-units share similar functional groups such as carboxylic acids and alpha-amino functions for sub-units (amino acids) of peptides and a base, sugar and a phosphate-groups for sub-units (nucleosides) for oligonucleotides.

The synthesis protocol of choice for the production of oligonucleotides and peptides is a solid phase synthesis, where the oligonucleotide or peptide is covalently linked to a solid phase. Nucleosides or amino acids (or any residues thereof) are stepwise coupled to a continuously growing oligonucleotide or peptide covalently linked to a solid phase. The solid phase synthesis comprises reaction steps of a heterogenous chemical reaction comprising a liquid phase and a solid phase where the reactant (nucleoside or amino acid) is provided in the liquid phase while the peptide or oligonucleotide is covalently bound to the solid phase.

The solid phase may be in particulate form and suitably dispersed in the liquid phase. The solid phase may be compressible and may range from a highly solvated polymeric resin to essentially incompressible particles or rigid structures with a high surface area. Moreover, the solid phase synthesis may be operated in batch, continuous mode, or a combination of both.

The solid phase synthesis protocols applied for both oligonucleotide and peptide synthesis include re-current cycles comprising a number of distinct procedural steps typically including deprotection, i.e. removal of protective groups, coupling reactions where an additional sub-unit (nucleoside or amino acid) is covalently linked to a growing polymer of a peptide or oligonucleotide, and steps comprising the displacement of by-products, reactants and solvents.

A cycle for the synthesis of a peptide usually comprises the steps of deprotection typically signifying that the protection group linked to the N-terminal α-amino group of the peptide-resin residue is cleaved leaving an unprotected α-amino functionality. In the subsequent step the carboxylic acid moiety of an N-terminal α-amino protected amino acid is covalently linked to the unprotected α-amino functionality of the peptide-resin residue under the formation of an amide coupling. Byproducts and excess reagents are removed after successful deprotection and coupling by washing procedures.

Solid phase oligonucleotide synthesis usually implements the phosphoramidite synthesis method comprising the use of phosphoramidite nucleoside building blocks (sub-units) which may be derived from protected 2′-deoxynucleosides (dA, dC, dG, and T), ribonucleosides (A, C, G, and U), or chemically modified nucleosides, e.g. LNA or BNA. To obtain the desired oligonucleotide, the building blocks are sequentially coupled to the growing oligonucleotide chain in the order required by the sequence of the product. By using nucleoside phosphoramidites instead of naturally occurring nucleotides the selectivity and yield is dramatically improved. The re-current cycle usually includes the steps of de-blocking or detritylation, coupling, capping (or sulphurization) and oxidation.

The solid phase used in oligonucleotide synthesis is usually non-swellable or low-swellable solid phases (supports). Suitable solid phases for oligonucleotide synthesis are porous silicates such as controlled pore glass (CPG). Recently, non-silicate solid phases have been introduced in SPOS such as polystyrene based resins including macro-porous polystyrene (MPPS) which tend to change volume as process conditions change (e.g. solvents and growth of oligonucleotide).

The heterogeneous chemical reaction may be operated in continuous or batch mode.

The present invention relates to the displacement of compounds when a reaction step of a heterogeneous liquid-solid phase chemical reaction protocol is considered to have reached a predefined reaction endpoint.

A reaction endpoint is deemed to be a point where the reaction has reached a useful yield. The compounds to be displaced are any compounds which are unfavorable for the next reaction step. Such unfavorable compounds may include excess reactants, by-products and solvents.

The displacement liquid reduces and suitably eliminates compounds deemed unfavorable for in the next reaction step. Hence, the word displacement should be interpreted to include any physical phenomenon causing a reduction of unfavorable compounds. Thus, a displacement procedure comprising the addition of a displacement liquid to a solid phase embraces phenomena such as reduction, removal, washing, percolation.

As elaborated the process is specifically configured to be associated with reaction steps of recurrent cycles of heterogeneous liquid-solid phase chemical reaction protocols for the formation of a target product which is synthesized by the consecutive introduction of similar sub-units. The process is suitably implemented in the same reaction chamber/vessel where also the reaction steps of the cycles are performed. Hence, the reaction chamber typically comprises the features needed to perform any one of the reaction steps of a cycle for the synthesis of the target molecule and may also contain elements specifically for the instant process. Thus, one benefit with the instant process is that it is implemented in the same reaction chamber where the reaction steps are performed, which reduces process complexity.

According to an aspect, valid for the most generic definition of the invention, the process encompasses the continuous removal the liquid phase from the reactor through the outlet and the discontinuous feed of a displacement liquid through the inlet to the chamber, and wherein the height of the liquid above the solid (h) is fluctuating over time with the proviso that the height (h) is never zero.

According to a further aspect, the reactor of the process comprises an inlet, an outlet, a liquid phase, a solid phase and means preventing the solid phase from escaping from the reaction chamber when liquid phase is removed from the reactor; continuously removing the liquid phase from the reactor through the outlet; wherein a displacement liquid is discontinuously feed through the inlet to the chamber, and wherein a continuous layer of liquid phase is maintained over the solid phase.

According to an aspect, the target compound is selected from peptides and oligonucleotides and the sub-units are selected from amino acids and nucleosides and any derivatives thereof.

In the context of the present invention an amino acid can be any organic compound comprising an amine and a carboxylic acid. One groups of amino acids comprise an α-amine and a carboxylic acid. Amino acid and amino acid derivative has herein the same meaning and can be used interchangeably. Important amino acids are the proteinogenic amino acids (often referred to as natural amino acids) which are incorporated biosynthetically into proteins during translation. Proteinogenic amino acids may be chemically modified.

A nucleoside in the context of the present invention can be any organic molecule conducive for the synthesis of a polynucleotide chain. Nucleoside derivatives include organic molecules comprising any one of derivatives of nucleobase (nitrogenous base) and/or derivatives of sugars or moieties replacing the sugar.

Peptides (also referred to as polypeptides) are molecules comprising at least 2 amino acids or derivatives of amino acids. At some point governed by the number of amino acids the molecule is denoted as a protein and not a peptide. Usually, a molecule having less than 100 amino acids (or amino acid derivatives) is denoted as a peptide.

Amino acids and derivatives thereof also encompass amino acid fragments. An amino acid fragment can be any number of fragments of a target peptide comprising from two amino acids up to fragment with a number of amino acids equaling the number of amino acids of the target peptide minus one amino acid, all fragments adding up to the target peptide.

Amino acids and derivatives thereof also comprise non-proteinogenic amino acids as well as any non-amino acid compounds which may enhance the utility of the target peptide for biological applications.

According to yet a further aspect, the target compound is selected from peptides and the sub-units are selected from amino acids and derivatives thereof. Amino acid and derivatives thereof may also include sub-units which would not fall under the traditional definition of an amino acid.

According to an aspect, the invention relates to a process for displacing compounds (and/or solvents) comprised in a liquid and solid phase of reaction steps of a solid phase peptide synthesis, the process comprising providing a reactor where the reaction steps occur, the reactor comprising a liquid and solid phase, means preventing the solid particles from escaping from the reactor when the liquid phase is removed from the reactor, continuously removing the liquid phase from the reactor while displacement liquid is discontinuously (intermittently) feed to the reactor, and wherein a continuous layer of liquid phase is maintained over the solid phase. According to an aspect, the invention relates to a process for displacing compounds (and/or solvents) comprised in a liquid and solid phase of reaction steps of a solid phase peptide synthesis, the process comprising providing a reactor where the reaction steps occur, the reactor comprising a liquid and solid phase, means preventing the solid particles from escaping from the reactor when the liquid phase is removed from the reactor, means for facilitating mass-transfer between the liquid and solid phase, continuously removing the liquid phase from the reactor and (while) displacement liquid is discontinuously (intermittently) feed to the reactor, and wherein a continuous layer of liquid phase is maintained over the solid phase.

A further aspect of the process comprises the sedimentation of the solid phase thereby forming a visual and by suitable sensors measurable phase boundary between the liquid and solid phase. Sedimentation of the solid phase will occur when agitation of the solid and liquid phase by a means for the facilitation of mass-transfer between the liquid and solid phase, e.g. by a stirrer, vortex mixing, nitrogen bubbling or recirculation pump, is interrupted. Thus, a further aspect of the process is that the continuous removal of the liquid phase from the reactor and the discontinuous feed of the displacement liquid to the reactor is implemented the means for the facilitation of mass-transfer between the liquid and solid phase, e.g. by a stirrer, vortex mixing, nitrogen bubbling or recirculation pump, is interrupted. Sedimentation of the solid phase infers that homogenization of the liquid and solid phase occurs in a reaction step preceding the displacement operation by the application of a means for the facilitation of mass-transfer between the liquid and solid phase, e.g. by a stirrer, vortex mixing, nitrogen bubbling or recirculation pump.

According to yet a further aspect the reactor deployed is a tank reactor (e.g. stirred tank reactor) or a column reactor.

According to a further aspect the reactions steps of a cycle are all performed in the reactor also used for displacing compounds of the present invention. If the process of a reaction step comprises the provision of a solid phase in particulate form evenly distributed in the liquid phase, homogenization by means for the facilitation of mass-transfer between the liquid and solid phase, then the application of the process for displacement of the invention mimics a fixed bed reactor while still using one and the same reactor.

A further aspect relates to a process comprising the washing of a solid phase by percolation, the solid phase comprised in a reactor, the process comprising continuously removing a liquid phase from the reactor and feeding a displacement/washing liquid discontinuously to the reactor while maintaining a continuous layer of a liquid phase over the solid phase.

In a further aspect the displacement liquid is introduced into the reactor any time after the means for facilitating the mass-transfer between has been terminated (not more engaged). Preferably, the displacement liquid is introduced when the height of the liquid phase above the solid phase has reached a pre-determined minimum threshold (h(min)).

According to an aspect, the liquid phase is removed from the reactor using a protocol which provides a superficial velocity of the displacement liquid over the solid phase not fluctuating more than about 30%, not more than about 20%, not more than about 10%.

According to a further aspect, the liquid phase is removed continuously from the reactor suitably providing that the superficial velocity of the displacement liquid over the solid phase not fluctuating more than about 30%, not more than about 20%, not more than about 10%.

According to an aspect, the solid phase is selected from silica containing materials and polymeric materials comprising reactive sites which are capable of covalently link the polymeric material (solid phase) with a (proximal) sub-unit, optionally through a linker, of the target compound (nascent compound).

According to yet a further aspect, the solid phase is selected from cross-linked polystyrene-based polymers comprising reactive sites capable of covalently bind the polystyrene-based polymers (solid phase) with a (proximal) sub-unit, optionally trough a linker, of the target compound (nascent compound).

### DESCRIPTION OF THE FIGURES

FIG. 1 illustrates a schematic reactor for the implementation of the process of the invention.

FIG. 2 illustrates the height of the liquid phase as a function of time.

### DETAILED DESCRIPTION OF THE INVENTION

An important feature of the invention is to dissociate the feed of displacement liquid to the reactor and the withdrawal of the liquid phase from the reactor, i.e. inlet and outlet flow of the reactor. Thus, the continuous output flow rate is adjusted for attaining a desired superficial velocity of the liquid phase over the solid phase while the inflow is adjusted in a discontinuous manner. The inflow of displacement liquid is adjusted in a manner that there is always a layer of liquid phase over the solid phase.

According to an aspect the liquid phase from the reaction chamber through the outlet is continuously removed while a displacement liquid is discontinuously feed through the inlet to the reaction chamber, whereby a continuous layer of liquid phase is always maintained over the solid phase preferably until a predetermined displacement endpoint is reached.

The displacement endpoint is defined as a predetermined amount of any compound or compounds which is/are detrimental for the next reaction step. In solid phase peptide synthesis, a deprotection agent is used for deprotecting the protected amino acid (most) distal to the resin (solid phase). It is important to remove the deprotection agent prior to the subsequent coupling step to an amount eliminating or minimizing the formation of non-target peptides. When the α-amine is Fmoc protected the deprotection agent may be piperidine. Hence, a displacement endpoint may be the defined as the amount of piperidine.

According to another aspect, the gas-liquid phase boundary and the liquid-solid phase boundary are determined thereby establishing the height (h) of the liquid phase over the solid phase, and suitably providing that the height (h) is never zero.

The height (h) is suitably fluctuating over time, preferably up to about 1000%, preferably up to about 500%, more preferably up to about 300%, more preferably up to about 200%.

As the displacement liquid is fed discontinuously to the reaction chamber the flowrate of the displacement liquid is typically higher than the flowrate of the purged liquid phase to provide that the solid phase is always covered by a layer of liquid phase.

According to an embodiment the feed of the displacement liquid is activated when the height of the liquid phase over the solid phase has reached a predetermined minimum, h(min). The feed of the displacement liquid is terminated when the height of the liquid phase over the solid phase has reached a predetermined maximum, h(max).

The height of the liquid phase may be monitored visually by an operator, but preferably, the height is provided by at least one sensor monitoring the gas-liquid phase boundary and the liquid-solid phase boundary. By suitable algorithms the height h of the liquid layer can be derived from the two-phase boundaries.

Preferably, the minimum height h(min) is selected such that the distribution of the displacement liquid on the liquid phase does not disturb the solid phase.

According to an aspect the inner cross-section area of the reactor is from about 10 cm² up to about 80000 cm².

According to a further aspect, the minimum height of the liquid phase h(min) above the solid phase (resin bed) is about 0.5 cm, preferably about 1 cm, preferably about 2 cm, about 3 cm, about 4 cm, about 5 cm. The minimum height h(min) may range from about 0.5 cm up to about 20 cm, suitably from about 1 cm to about 10 cm. The maximum height h(max) is preferably about 2 cm, preferably about 4 cm, preferably about 5 cm, or about 6 cm, or about 7 cm, or about 10 cm, or about 20 cm. The maximum height h(max) of the liquid phase is suitably in a range of from about 2 cm up to about 20 cm, suitably from about 4 cm up to about 10 cm. Another metric for the height of the liquid level is the arithmetic mean of the height. If h(min) is 1 cm and h(max) is 2 cm the mean height, h(mean) is (1+2)/2=1.5. According to an aspect the mean height h(mean) of the liquid phase is in the range of from 1.0 cm up to around 10 cm.

According to an aspect, the ratio between h(min) and h(max) [ratio: h(max)/h(min)] is between about 1.1 and about 6.

According to an aspect, the superficial velocity of the displacement liquid (over the solid phase) in the reactor is held in a range of from about 100 cm/hour up to about 400 cm/hour, preferably from about 150 cm/hour up to about 300 cm/hour, preferably from about 175 cm/hour up to about 250 cm/hour. By superficial velocity is herein meant a hypothetical linear average velocity given by dividing the volumetric flow rate [volume/time] by the cross-sectional area of the solid phase (fixed bed) [area]. The superficial velocity may also be denoted as the percolation velocity. The percolation velocity is suitably adjusted with account to the mass transfer kinetics of the compounds to be displaced from the solid phase (resin), and of the pressure drop of the solid phase (bed of the solid phase in particulate form dispersed in the liquid phase: resin bed).

The rate of the outflow of liquid phase from the reaction chamber is continuous and preferably is not allowed to fluctuate more than 30%, preferably not more than 20%, suitably not more than 10%. The out-flow rate is typically regulated to provide a superficial velocity in the range specified herein.

According to an aspect, the in/out flow ratio (ratio of flowrate of displacement liquid to flowrate of removed liquid phase) is from above 1 and suitably from about 5 up to about 10.

When the level reaches the low threshold, the solvent is added by portion of a defined volume. Once this volume has been added, the inflow is stopped and the liquid level is measured, which can be done under optimal conditions, as the sensor beam or visual inspection of the level is not disturbed by the flow of the input solvent. The percolation (outflow) causes this level to decrease and when the low level is reached again, the feed pump is reactivated.

One embodiment of the invention relates to a solid phase synthesis comprising a solid phase in particulate form dispersed in the liquid phase where the reaction step of a cycle is conducted when the solid phase is homogeneously dispersed in the liquid phase. One can say that the solid phase is homogenized by a means for the facilitation of mass-transfer between the liquid and solid phases such as a stirrer, vortex mixing, nitrogen bubbling or recirculation pump. Hence, the reactor must comprise means for the facilitation of mass-transfer between the liquid and solid phases in order to homogenize the solid phase in the liquid phase during a reaction step. Usually, a solid phase synthesis comprising reaction steps, said steps comprising homogenization of the solid phase, comprises a reactor which is operated in batch mode.

When the reaction of a reaction step such as deprotection or coupling steps has reached a predetermined reaction endpoint it may be preferred to let the reaction composition comprising a liquid phase and a solid phase (homogenized reaction composition) sediment until a phase boundary specifically between the liquid and solid particles is detectable or observable.

Preferably, before the displacement (washing) liquid is fed to the reaction chamber the means for the facilitation of mass-transfer between the liquid and solid phases and homogenizes the solid phase (if present) is interrupted and the reaction composition is allowed to sediment until the liquid and solid particles have separated to the extent it is possible to visually monitor or by means of at least one detector to monitor the gas-liquid phase boundary and the liquid-solid particle boundary.

It is preferred that there is always a liquid layer over the solid phase at the moment the displacement liquid is introduced which prohibits that the solid phase is drying out.

However, the displacement liquid may also be introduced any time after the means for facilitating mass-transfer has been discontinued. The displacement liquid may also be introduced before the means for facilitating mass-transfer has been discontinued. The displacement may also be introduced into the reactor after the means for facilitating mass-transfer has been discontinued but before a visual or measurable (detectable) phase boundary between the liquid and solid phase has been established.

In principle there is no boundary between the liquid phase and the solid phase in the strict meaning of the term phase boundary. Rather, the solid phase, or rather solid particles, is/are always immersed in the liquid phase or dispersed in the liquid phase. Thus, the term solid phase should be understood as a solid phase in particulate form dispersed in a liquid phase. If the reaction composition is not subjected to forces trying to evenly distribute the solid particles in the liquid phase the particles will by gravity gradually sink whereby a liquid layer on top of the dispersion of solid particles is formed. After some time there will be an equilibrium between gravity and repulsive forces between the solid particles providing a constant level of the dispersion comprising the solid particles, i.e. level of the solid phase at a liquid solid phase boundary. At this equilibrium one can denote the visually observable and detectable transition between the liquid phase and the dispersion as a quasi phase boundary. Here this quasi boundary is for simplicity denoted as the liquid-solid boundary.

The solid particles dispersed in the liquid phase of the heterogeneous liquid-solid phase chemical reaction of the invention may also be referred to as a bed of solid phase in particulate form or simply resin bed.

One of the characteristic features of the invention is the provision of a discontinuous or intermittent feed of displacement/washing liquid and a continuous outflow of liquid phase. By the dissociation of the inflow of washing liquid and outflow of liquid phase, the flowrate of the washing liquid can vary while maintaining an essentially constant superficial flow velocity (or simply superficial velocity) of the liquid phase over the bed of solid particles. Herein the superficial velocity is defined as follows:

$${u}_{s} = \frac{Q}{A}$$

where:
uₛ denotes the superficial velocity of a given phase
Q denoted the flow rate of the phase
A denotes the cross-sectional area

The present process may also be contemplated as a percolation process, i.e. an extraction procedure where soluble constituents are removed from the solid phase by extraction by means of the feed of the displacement liquid.

A further aspect where the process further comprises the sedimentation of the solid phase thereby forming a gas-liquid phase boundary, a liquid-solid phase boundary and a liquid phase over the solid phase.

According to a further aspect, the invention comprises a continuous removal of the liquid phase from the reactor through the outlet wherein a displacement liquid is intermittently (discontinuously) feed through the inlet to the reactor, and wherein the height (h) of the liquid layer above the solid layer is fluctuating over time with the proviso that the height (h) is never zero.

According to a further aspect, the sedimentation is forced by continuous outflow of liquid phase. If the reaction composition is not subjected to forces trying to evenly distribute the solid particles in the liquid phase the particles will by gravity gradually sink whereby a liquid layer on top of the dispersion of solid particles is formed. The outflow of the liquid phase will force the sedimentation and after some time there will be an equilibrium between gravity and repulsive forces between the solid particles providing a constant level of the dispersion comprising the solid particles, i.e. level of the solid phase at a liquid solid phase boundary. At this equilibrium one can denote the visually observable and detectable transition between the liquid phase and the dispersion as a quasi-phase boundary. Here this quasi boundary is for simplicity denoted as the liquid-solid boundary.

According to yet a further aspect, the discontinuous percolation washing is implemented in the reactor where all reaction steps of a cycle of a heterogeneous chemical reaction are performed.

### Reactor

The reactor, or reactor chamber, preferably is a reactor for the implementation of a heterogeneous liquid-solid phase chemical reaction protocol for the formation of a target molecule which is synthesized by the consecutive introduction of sub-units. More specifically, the reactor may be any reactor suited for solid phase peptide synthesis and solid phase oligonucleotide synthesis. According to an aspect the reactor may be any reactor applicable for solid phase peptide synthesis. The reactor may be a reactor for batch mode or for continuous mode, alternatively a reactor which can be operated in both batch and continuous mode.

Preferably, the reactor is selected from column reactors and tank reactors. Reactions with an unfavorable reaction kinetics may be implemented in a tank reactor and suitably in batch operation. Reactions with a favorable reaction kinetics may be implemented in a column reactor suitably in continuous operation.

The reaction kinetics for the reaction steps, specifically rate limiting reaction, in oligonucleotide synthesis are such that a column reactor is preferred. The column reactor for solid phase oligonucleotide synthesis is usually operated in continuous mode and may have a configuration enabling the re-circulation of the reaction solution

The reaction kinetics of the reaction steps in SPPS, specifically the rate limiting coupling step (where the amide binding is formed), are such that a tank reactor is preferred. The tank reactor for SPPS is generally operated in batch mode.

The reactor may comprise feed-back systems capable of re-circulating the reaction solution.

According to an aspect the reactor is a batch type reactor. The reactor is suitably a tank reactor (such as a stirred tank reactor) preferably specified for batch operation. Suitably, the reaction chamber comprises at least one inlet, one outlet, a liquid phase, and a solid phase. The reaction chamber may also comprise a means for facilitating the mass transfer between the liquid and solid phase and means enabling the removal of the liquid phase without depletion of the solid phase. The reaction chamber is suitably equipped with a displacement liquid distribution system.

Means for facilitating the mass transfer between the liquid and solid phase may be a mechanical device acting on the liquid comprising the solid phase, the introduction of irregular disturbances or turbulent motion of the liquid and solid phase. Examples of mechanical devices include impellers such as anchor, propeller, flat blade disc-turbine, paddle, gate anchor and helical screw. Increased mass-transfer may also be achieved by way of a gaseous phase such as nitrogen bubbling, vortex or recirculation.

The displacement liquid is suitably distributed to the liquid phase on the solid phase by a suitable liquid distribution system. The displacement liquid is fed to the liquid phase such that the solid phase is essentially capable of providing a plug flow of the displacement liquid over the solid phase. The displacement liquid is preferably distributed to the liquid phase such that disturbances of the solid phase are minimized. The flowrate of the displacement liquid into the reactor is to an extent governed by the type of distribution system inside the reactor. The distribution system may be configured to comprise nozzles capable of particularizing the liquid into a spray. The nozzles of the distribution system may be configured to be able to modulate the particle size of the liquid. If after a reaction step solid particles are present on the walls of the reactor above the liquid phase it may be advisable to distribute the displacement liquid in a physical form facilitating the removal of solid particles from the wall. After the reactor walls have been cleaned the particle size of the liquid is preferably reduced by regulating the nozzles thereby forming a fine spray not disturbing the solid phase. Suitably, inlet flow rate of the displacement liquid should be sufficient for the distribution system to function correctly, to ensure a homogeneous distribution of the fluid, and to allow adequate rinsing of the reactor walls to eliminate traces of compounds and solid phase particles. The operation of nozzles often requires a restricted flow range to ensure optimal operation. It is therefore important to be able to set the inlet flow rate within an optimal range, suitably dependent on the size of the distribution system, and therefore to make the inflow independent of the superficial velocity (percolation velocity) constraints. The superficial velocity is highly correlated to the outlet flow rate of the liquid phase.

The reactor is suitably a glass, stainless-steel, hastelloy or coated reactor with a preferred capacity from about 0.5 up to about 5000 liters. A filtration device is typically placed at the bottom of the reactor for retaining the solid phase (resin) while discharging the liquid phase. This filtration device may be formed of a sintered stainless-steel material but, could consist of a porous polymer or filtering sheet or any other filtration system known to a person skilled in the art.

According to an aspect, the reactor may have at least one inlet, for the introduction of the resin, the reagents and the solvents used for the different reaction steps. It is preferred that liquids are introduced by one or more self-priming pumps. The introduction flow rate, as well as the volume introduced are suitably measured and quantified by mass flow sensors. Any liquid fed to the reactor may be heated or cooled as needed before entry into the reactor via a heat exchanger.

According to an aspect, the reactor may be configured to enable the real-time measurement of the evolution of the chemical species in the discharged liquid from the reactor via measuring cells which may be selected from conductivity cells, a near-infrared cells (e.g. from 1 mm to 30 mm of optical path), UV cells (e.g. from 0.5 mm to 10 mm of optical path) and refractive index cells.

As alluded to before the gas-liquid phase boundary and the liquid-solid phase boundary may be monitored visually or by at least one sensor. By the monitoring of these two phase-boundaries the height of the liquid phase over the solid phase can be provided. Suitable sensors are sensors emitting ultrasound or electromagnetic radiation in the UHF and microwave length (radar sensors) and lasers (including lidar sensors). By a suitable sensor or sensors the height (h) of the liquid phase above the solid phase. According to an aspect, two sensors (detectors) are implemented one dedicated for monitoring the liquid-solid phase boundary the other dedicated for monitoring the gas-liquid phase boundary. Preferably, of two sensors are implemented both simultaneously monitor the two phase boundaries. According to a further aspect, one sensor is implemented which is capable of monitoring both phase boundaries. The detection of the liquid-solid phase boundary and the gas-liquid phase boundary facilitates the regulation of the discontinuous displacement liquid fed to the reaction chamber.

Further to the application of sensors for monitoring the height of the liquid phase, the reactor may be equipped with a variety of sensors including sensors for measuring pressure, conductivity and pH.

A further advantage with the present process with the provision of a discontinuous feed of displacement liquid to the reaction is that the monitoring of the phase boundaries and the height of the liquid phase above the solid phase can take place when no displacement liquid is fed to the reactor. The measurement of the height of the liquid phase during a period where no displacement liquid is feed to the reactor provides for a more accurate measurement of the phase boundaries and hence the height of the liquid phase. The liquid surface is not influenced by the displacement liquid. Also, sensors are not disturbed by liquid particles in the gas phase (pray of displacement liquid from distribution system (system of nozzles). Also, discontinuous operation of the feed of the displacement liquid increases the freedom of regulating the flow rate of the displacement liquid. Thus, the flow rate of the displacement liquid can be modulated. For example, periodically the flow rate of the displacement liquid can be decreased. Also, the duration of the feed of displacement liquid may be modulated.

Thus, both flow rate and duration of the feed of displacement liquid to the reactor may we varied. The flow rate may temporarily be increased within a delivery phase/period or between delivery phases. If there is a need for decreasing the flow rate of the displacement liquid the duration may be increased so that a constant volume of displacement liquid is fed to the reactor during each delivery phase.

According to an embodiment the process is implemented in a solid phase peptide synthesis protocol. In particular, the process may be implemented after any of the reaction steps of a cycle (or between two reaction steps), more specifically after the deprotection step and after the coupling step.

### Description of Embodiments Practiced in the Examples

A di-peptide is synthesized using a solid phase peptide synthesis protocol (SPPS) and Fmoc protection groups for the α-amide. A 4-Methylbenzhydrylamine Resin hydrochloride (MBHA-resin) with a loading of 1.12 meq/g is used as the solid phase. The MBHA-resin is loaded in a stirred tank reactor (30 cm diameter) having a filter bottom in form of a sintered stainless-steel material and washed with dimethylformamide (DMF). The reactor is equipped with a stirring blade. On the top of the reactor, several inlets are present, of which one inlet is dedicated to the introduction of the resin and one inlet is dedicated to the introduction of the solvents (DMF, piperidine, etc), introduced by a self-priming pump. The introduction flow rate, as well as the volume introduced are measured and quantified by a mass flow sensor. The flow rates can range from 35l/h to 600l/h (50 to 850 cm/h). For cleaning the reactor correctly between each step of the synthesis, a device for dispersing the solvent such as the displacement (washing) liquid is located at the end of the line, at the level of the reactor. This device operates correctly between 20 and 1000l/h. An inlet pipe dedicated to the introduction of the agent for deprotecting the amino acid can also be placed on the top of the reactor. It is done thanks to a self-priming pump, of which the flow rate is 20 to 1000l/h (28 to 1415 cm/h). The introduction flow rate, as well as the volume introduced are measured and quantified by a mass flow sensor. An inlet pipe for additional solvent, of which the volume and the flow rate are controlled by a mass flow sensor, can also be placed at the top of the assembly reactor.

The resin is neutralized by the addition of 10% solution of N, N-Diisopropylethylamine (DIEA) in DMF. Subsequently, an Fmoc protected Rink amide linker is added to the reactor and the linker is coupled to the resin using DIC/OXYMA. After successful coupling the resin is washed in DMF.

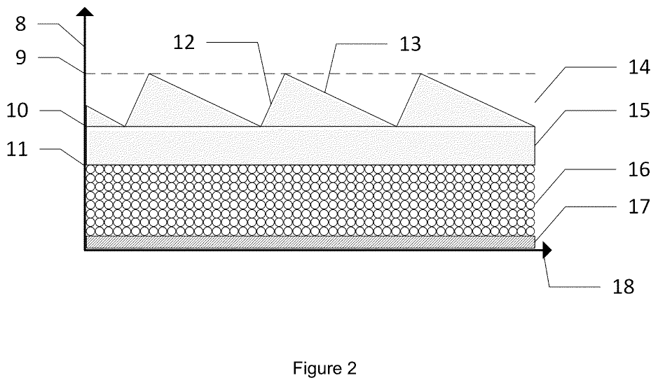

The first recurrent cycle comprises the deprotection of the Fmoc group by the addition of a 25% piperidine/DMF solution. After deprotection the resin in washed using DMF and the process of the instant invention (discontinuous percolation washing). After successful deprotection the stirring is discontinued, and the resin (solid phase) is allowed to sediment to form a resin bed and a liquid phase above the bed thereby forming a resin bed which is distributed homogeneously and horizontally in the reactor. Washing solvent is introduced homogeneously on the surface of the liquid and the reactor wall to remove all traces of piperidine, by-products (as dibenzofulvene) and solid phase. The solvent is introduced when the low-level liquid threshold h(min) of 1 cm is reached. The reactor is continuously drained during the percolation wash. The percolation wash is performed until reaching the desired endpoint of 100 ppm of piperidine. FIG. 2 illustrates the discontinuous flow protocol of the washing liquid with the height of the liquid phase as a function of time.

After successful deprotection and after the discontinuous washing, a Fmoc protected amino acid (Fmoc-AA-OH) is introduced into the reactor. The coupling of the Fmoc protected amino acid is conducted with DIC/OXYMA in DMF (0.5 M). After coupling the stirring is discontinued and the resin (solid phase) is allowed to sediment to form a resin bed and a liquid phase above the bed thereby forming a resin bed which is distributed homogeneously and horizontally in the reactor. The washing solvent is introduced homogeneously on the surface of the liquid and the reactor wall to remove all traces of reactant (Fmoc-AA-OH), coupling agents (DIC/OXYMA) and by-products (as diisopropylurea). The washing solvent is introduced discontinuously at 600 l/h when the low-level liquid threshold h(min) of 3.0 cm is reached. The reactor is continuously drained during the percolation wash at a superficial speed of 212 cm/h (150 l/h).

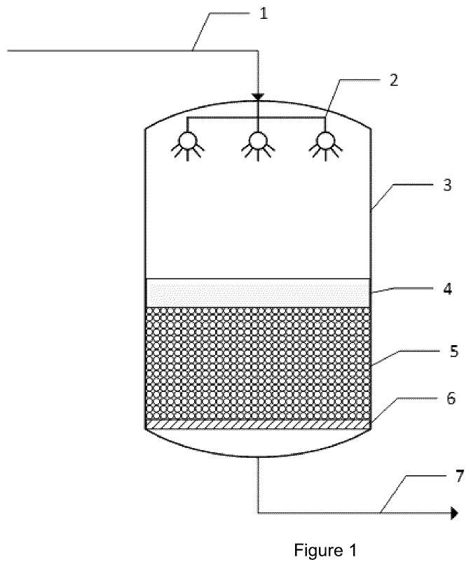

FIG. 1 illustrates a schematic reactor for the implementation of the process of the invention. The reactor comprises an inlet (1) and a solvent distribution system (2). Through the inlet and the solvent distribution system the displacement liquid is fed to the reactor. At the bottom of the reactor an outlet (7) is present for the discharge of the liquid phase. In the reactor a gas phase (3), a layer of a liquid phase (4) over the solid (resin) phase (5) is presented. A filtration device (6) is configured at the bottom of the reactor enabling the discharge of the liquid phase through the outlet (7) without removing the solid phase.

FIG. 2. illustrates the height of the liquid phase over the resin as a function of time. Also included schematically are the solid (resin) phase layer (16) and the filtration device (17) as well as the liquid phase, e.g. (15). (8) denoted the level axis and (18) is the time axis. (9) is the high-level liquid threshold [h(max)] and (10) is the low-level liquid threshold, h(min). The triangle (12) specifies the duration of the displacement liquid fed to the reactor and the increase of the level of the liquid phase over time. The start of the inflow of the displacement liquid is triggered by the liquid phase reaching the low-level liquid threshold [h(min)] (10). The inflow of displacement liquid is stopped once the level of the liquid phase has reached the high-level threshold h(max) (9). The inflow of displacement liquid remains inactive until the liquid phase has reached the low-level liquid threshold, h(min) (10). The triangle (13) specifies the duration during which the displacement liquid is shut off and the decrease of the liquid phase over time. (14) denotes the variable layer of the liquid phase during discontinuous percolation. (11) denotes the resin bed level and consequently the liquid-solid phase boundary.

Outline of the Discontinuous Percolation Washing Performed in a Stainless-Steel Reactor with a Diameter of 30 cm

Firstly, the resin bed level (after draining) is measured using the level sensor and recorded. This step is performed after draining the reactor:
Before the first deblocking, or
Before last deblocking (after draining the penultimate deblocking solution).
Before the coupling

After the deblocking/coupling step, the stirrer stops at a defined position to avoid an interference with the radar probe and allow level measurement during percolation step. For this, a sensor is installed in the agitator to assure the defined position.

Percolation starts directly after deblocking/coupling step without draining the piperidine/coupling solution.

The valve in the bottom of the reactor is opened and the draining pump started at a flow rate set by the user. The draining is done continuously at a fixed outflow during the whole percolation sequence.

When the level sensor reaches the low-level liquid threshold h(min), the introduction valve opens, and the introduction pump starts to introduce a volume defined by the user. The inflow must be higher than the outflow to ensure efficient fluid distribution with the nozzles and washing out the piperidine/coupling solution.

The level setpoint above the resin bed is very important, under no circumstances should the solvent level fall below the level of the resin bed. Otherwise the resin may dry out and the quality of the percolation may be affected.

This cycle is repeated until the stop criteria chosen by the user is reached, as below either online monitoring:
FT-NIR threshold
UV threshold

Or offline monitoring:
Chloranil test
pH
Spectrometric analysis (e.g. DNFB)

Once the end of percolation setpoint is reached, the draining stops and the DMF introduction pump stops (if an introduction is in progress).

The homogenization is performed immediately after percolation and without draining the washing solution from the reactor. Afterwards, the residual concentration (piperidine or coupling solution) is analyzed by an online or offline method (DNFB, chloranil), and this quantification will define if it is necessary to make an extra batch washing to reduce residual concentration or to start to the next step.

Discontinuous Percolation Washing Performed Using a Glass Reactor with a Diameter of 6 cm

Firstly, the resin bed level is measured manually without draining the reactor.

Percolation starts directly after deblocking/coupling step without draining the piperidine/coupling solution.

The valve in the bottom of the reactor is opened and the draining pump started at a flow rate set by the user. The draining is done continuously at a fixed outflow during the whole percolation sequence.

When the level reaches the low-level liquid threshold h(min), the introduction pump starts to introduce the solvent until reaching a maximum height h(max). The inflow must be higher than the outflow to ensure that the solvent level do not fall below the level of the resin bed. Otherwise, the resin may dry out and the quality of the percolation may be affected.

This cycle is repeated, and the washing solution is collected in different fractions that are analyzed by an offline method (DNFB, chloranil) to determine the residual concentration at the reactor outlet.

Once the end of percolation setpoint is reached, the draining stops.

The homogenization is performed immediately after percolation and without draining the washing solution from the reactor. Afterwards, the residual concentration (piperidine or coupling solution) is analyzed by an offline method (DNFB, chloranil), and this quantification will define if it is necessary to make an extra batch washing to reduce residual concentration or to start to the next step.

### EXAMPLES

### Overview

Example 1 relates to the comparison of the discontinuous percolation washing of the invention with continuous percolation washing and batch washing. Here a glass reactor with a diameter of 6 cm is used which is loaded with a 4-Methylbenzhydrylamine hydrochloride resin (MBHA-polystyrene)

In examples 2 to 6 a di-peptide resin, Fmoc-Ala-Gly-Rink Amide-MBHA-Resin, is used as a model resin mimicking conditions of a solid phase peptide synthesis. Examples 2 to 5 proved conditions after a deprotection step of a cycle.

Example 6 provides conditions after a coupling step of a cycle.

Examples 2 and 6 relate to the comparison of batch washing and discontinuous percolation washing according to the invention.

Examples 3 to 5 present the impact of some parameters of the discontinuous percolation washing (superficial velocity, varying heights of liquid phase above solid phase, variation of maximum height of liquid phase above solid phase) on washing volume and time at a predefined endpoint.

### System Description

In the following examples two different reactors are used, a glass reactor having a diameter of 6 cm and a stainless-steel reactor having a diameter of 30 cm.

Both reactors are equipped stirred tank reactors (STR) comprising a stirring blade. Further, the reactors have a filtration device in the bottom for retaining the solid phase while discharging liquid. On the top of the reactors several inlets are present, of which one inlet is dedicated to the introduction of the resin and another inlet used for the introduction of various solvents such as synthesis solvent and displacement liquid. At the bottom of the reactors an outlet is present used for discharging liquids. The volume and flowrate of the solvents/liquids are measured by a mass-flow sensor. The reactors are further equipped with a distribution system which distributes the incoming liquid such that the wall of the reactor is cleaned. The stainless-steel reactor is also equipped with a sensor for measuring the phase boundaries and thereby the height of the liquid phase above the solid phase.

In each of the examples where discontinuous percolation washing and the batch washing is compared the same reactor is used.

In example 1 a glass reactor with a diameter of 6 cm is used. In all other examples a stainless-steel reactor with a diameter of 30 cm is used.

In all examples the same base resin (solid phase) is used: 4-Methylbenzhydrylamine hydrochloride resin (MBHA-polystyrene)

### Batch Washing Description

The batch washing step is carried out by successive batches constituting the introduction of the washing solvent, stirring for 5 minutes and then draining. The endpoint of the batch washing step after deprotection is determined by measuring the residual concentration of piperidine in the washing liquid by near IR quantification. After coupling the endpoint is established by measuring the absorbance of the residual coupling solution (e.g. coupling agent, amino acids and by products) in the washing liquid by UV quantification.

### Discontinuous Percolation Washing Description

The discontinuous percolation washing is carried out on a resin disposed as a fixed bed in a reactor equipped with a filtering bottom and distributed homogeneously and horizontally on its surface to avoid any preferential path of the washing solvent through the resin bed.

### Example 1

In this example discontinuous percolation washing of the invention is compared with continuous percolation washing and batch washing with respect to washing volume and duration to attain a predetermined endpoint defined as residual piperidine in the discharged liquid phase.

In this example the different washing operations are tested during conditions after a deprotection step of a cycle using a DMF/piperidine for deprotection of base labile α-amine protection groups (e.g. Fmoc).

A stirred tank reactor (STR) of glass with a diameter of 6 cm is loaded with a 4-Methylbenzhydrylamine hydrochloride resin of to a height of 6 cm. Subsequently, a deprotection agent, a DMF/piperidine (25%) solution, is added to the reactor and the resin is homogenized in the solution by stirring under 10 minutes. The solution is drained from the reactor and a deprotection agent, a DMF/piperidine (25%) solution, is added to the reactor and the resin is homogenized in the solution by stirring under 5 minutes. The deprotection solution is drained from the reactor in the case of batch washing. The homogenized resin with the deprotection solution is the starting point of the assessment of the two different percolation wash operations.

Discontinuous percolation: The stirrer is stopped, the resin bed is distributed homogeneously and horizontally in the reactor. The washing solvent (displacement liquid), DMF, is introduced homogeneously on the surface of the liquid and the reactor wall to remove all traces of the deprotection agent (piperidine) to be removed. The solvent is introduced to the reactor when the low-level liquid threshold h(min) of 1 cm is reached until a high-level liquid threshold h(max) of 3 cm is reached. The reactor is continuously drained during the percolation wash at a flowrate of 1.3 liter/hour to obtain a superficial velocity of 45 cm/h over the resin bed. The percolation wash is performed until reaching the desired endpoint of 100 ppm of piperidine.

Batch wash: The washing solution (DMF) is introduced homogeneously and stirred for 5 minutes. The reactor is then drained. The batch wash is repeated until reaching the desired endpoint of 100 ppm of piperidine.

Continuous percolation: The stirrer is stopped, the resin bed is distributed homogeneously and horizontally in the reactor. The washing solvent is introduced homogeneously on the surface of the liquid and the reactor wall to remove all traces of the agent (piperidine) to be removed. The solvent is introduced and drained continuously during the percolation wash at a superficial velocity of 45 cm/h and the liquid level is stable at 3 cm above the resin bed. Thus, the flowrate of the washing solvent equals the flowrate of the liquid phase discharged from the reactor. The continuous percolation wash is performed until reaching the desired endpoint of 100 ppm of piperidine.

The volumes of washing liquid (displacement liquid) and duration at a set endpoint for the three washing operations are presented in table 1.

TABLE 1
Washing efficiency according to the washing
method in a small-scale reactor

| | Washing | Washing duration |
|---|---|---|
| Washing method | volume (ml) | (min) |
| Batch wash | 1360 | 64 |
| Discontinuous percolation | 366 | 17 |
| Continuous percolation | 471 | 22 |

Examples 2 to 6: In all examples 2 to 5 the same MBHA-resin has been used as in example 1 yet with a di-peptide covalently linked to the resin by a Rink amide linker. The di-peptide resin, Fmoc-Ala-Gly-Rink Amide-MBHA-Resin, is produced according to a protocol presented by table 2. In example 6 a NH2-Gly-Rink Amide-MBHA-Resin is provided and the Fmoc-Ala-OH is coupled.

Table 2 describes the solid phase synthesis of a di-peptide linker resin that will be used for the examples 6 to 10.

TABLE 2
Overview of the SPPS procedure to provide
the Fmoc-Ala-Gly-Rink Amide-MBHA-Resin
MBHA-resin (Loading 1.12 meq/g)

| | | |
|---|---|---|
| Step 1: Solid phase assembling of the peptide Step 1a: Preparation of the resin and Coupling of Rink amide linker | [symbol] | 1. Washes with DMF 2. Resin neutralisation with 10% DIEA/DMF (4×) 3. Coupling of 1.5 eq Rink amide linker with 3 eq DIC/3 eq Oxyma in DMF (0.5M). 4. Coupling test (if the test fails, recoupling with Rink amide linker/DIC/Oxyma) 5. Washes with DMF |

Fmoc-Rink Amide-MBHA-Resin

| | | |
|---|---|---|
| Step 1b: Fmoc deprotection and coupling cycles (repeated 2 times) | [symbol] | 1. Treatment with 25% Piperidine/DMF (2×) 2. Washes with DMF 3. Coupling of 3 eq Fmoc-AA-OH with 3 eq DIC/3 eq Oxyma in DMF (0.5M) 4. Coupling test (if the test fails, recoupling with Fmoc-AA-OH/DIC/Oxyma) 5. Washes with DMF |

Fmoc-Ala-Gly-Rink Amide-MBHA-Resin

### Example 2

Piperidine washing efficiency as a function of the washing method.

In this example discontinuous percolation washing of the invention is compared with batch washing with respect to washing volume and duration to attain a predetermined displacement (percolation) endpoint defined as the residual piperidine in the discharged liquid phase.

Batch washing and discontinuous percolation washing are tested during conditions after a deprotection step of a cycle using a DMF/piperidine for deprotection of base labile α-amine protection groups (e.g. Fmoc).

A stainless-steel stirred tank reactor (STR) with a diameter of 30 cm and comprising a bottom filter is loaded with a Fmoc-Ala-Gly-Rink Amide-MBHA-Resin to a height of 10 cm. Subsequently, a deprotection agent, a DMF/piperidine (25%) solution, is added to the reactor and the resin is homogenized in the solution by stirring under 10 minutes. The solution is drained from the reactor and a deprotection agent, a DMF/piperidine (25%) solution, is added to the reactor and the resin is homogenized in the solution by stirring under 5 minutes. The deprotection solution is drained from the reactor in the case of batch washing. The homogenized resin with the deprotection solution is the starting point of the assessment of the percolation wash operation.

Discontinuous percolation: The stirrer is stopped, the resin bed is distributed homogeneously and horizontally in the reactor. The washing solvent DMF is introduced homogeneously on the surface of the liquid and the reactor wall to remove all traces of the deprotection agent (DMF/piperidine) to be removed. The washing solvent (1 liter of DMF) is introduced to the reactor at a flowrate of 600 l/h when the low-level liquid threshold h(min) of 3.0 cm is reached and is terminated at a high-level liquid threshold h(max) of 4.0 cm. The feed of washing liquid is discontinued for about between 20 to 25 seconds. The reactor is continuously drained during the percolation wash at a flowrate of 150 liter/hour representing a superficial speed of 212 cm/h over the resin bed. The percolation wash is performed until reaching the desired endpoint of 100 ppm of piperidine.

Batch wash: The washing solution (6 liters of DMF) is introduced homogeneously and stirred for 5 minutes. The reactor is then drained. The batch wash is repeated until reaching the desired endpoint of 100 ppm of piperidine.

TABLE 3
Washing efficiency according to the washing
method in a large-scale reactor

| | Washing | Washing duration |
|---|---|---|
| Washing method | volume (l) | (min) |
| Batch wash | 60.1 | 50 |
| Discontinuous | 25.9 | 11.3 |
| percolation | | |

### Example 3

Influence of the superficial velocity on the Piperidine washing.

The flow rate of the washing solvent is optimized according to the diffusion speed of the (species) of the piperidine in order to allow the transfer of the (species) piperidine to be removed from the solid phase (resin) to the liquid phase (washing solvent). Thus, a too high flow rate would imply an overconsumption of the washing solvent and an increase of the washing duration. Furthermore, if the outflow is too high, the piperidine may not diffuse fast enough to the liquid phase and after homogenization the piperidine content may increase. In this case, an additional batch wash is necessary to reach the final endpoint.

The same stainless-steel reactor as in example 2 is used. Furthermore, an Fmoc-Ala-Gly-Rink Amide-MBHA-Resin (height 10 cm) is loaded and homogenized with a DMF/piperidine solvent according to the protocol of example 2.

The stirrer is stopped and the resin bed is distributed homogeneously and horizontally in the reactor. The washing solvent (DMF) is introduced homogeneously on the surface of the liquid (resin) and the reactor wall to remove all traces of the agent to be removed. The solvent (1 liter of DMF) is introduced at 600 l/h when the low-level liquid threshold h(min) of 3.0 cm is reached and discontinued at a high-level liquid threshold as indicated in table 4. The feed of washing liquid is discontinued for about between 20 to 25 seconds. The reactor is continuously drained during the percolation wash at a flowrate generating the superficial velocities of table 4. The percolation wash is performed until reaching the desired endpoint of 100 ppm of piperidine

TABLE 4
Washing efficiency according to the
outflow in a large-scale reactor

| Superficial outflow | Washing volume | Washing duration | Hₘₐₓ Liquid |
|---|---|---|---|
| velocity (cm/h) | (l) | (min) | level (cm) |
| 212 | 25.9 | 11.3 | 4 |
| 424 | 26.8 | 10.0 | 3.5 |

In the case of percolation at 424 cm/h, the percolation was stopped after 5 minutes when the piperidine content was below 100 ppm (correspond to a consumption of 20.8 liters of DMF). However, after homogenization, the piperidine content increased due to a late diffusion from the solid to liquid phase. Consequently, an additional batch wash (6 liters of DMF, 5 minutes stirring) was performed to decrease the concentration below the desired endpoint. The superficial velocity washing liquid should be adapted to the rate of diffusion between liquid and solid phase. If the superficial velocity is too high with regard to the diffusion the endpoint is reached before a diffusion equilibrium is obtained, necessitating additional percolation or batch washing

### Example 4

The influence of the minimum liquid level above the resin on the piperidine washing efficiency.

The same stainless-steel reactor as in example 2 is used. Furthermore, a Fmoc-Ala-Gly-Rink Amide-MBHA-Resin (height 10 cm) is loaded into the reactor and homogenized with a DMF/piperidine solvent according to the protocol of example 2. The stirrer is topped and the washing solvent, DMF, is introduced homogeneously so as not to disturb the resin bed and so that it remains horizontal on its surface. The discontinuous solvent introduction is carried out when the low-level liquid threshold h(min) is reached in order to limit the remixing phenomena and thus optimize the washing step. In order to verify the influence of the liquid level above the resin, the flowrate of the washing solvent introduced into the reactor was the same as the flowrate of the liquid phase discharged from the reactor.

The resin bed is distributed homogeneously and horizontally in the reactor. The washing solvent (DMF) is introduced homogeneously on the surface of the liquid and the reactor wall to remove all traces of the agent to be removed. The washing solvent is introduced at a flowrate of 600 l/h when the low-level liquid threshold h(min) min is reached and discontinued at a high-level liquid threshold as indicated in table 5. The reactor is continuously drained at a superficial velocity of 212 cm/h corresponding to a flowrate of the discharged liquid phase of 150 liter/hour. The percolation wash is performed until reaching the desired endpoint of 100 ppm of piperidine.

TABLE 5
Washing efficiency according to the minimum
liquid level in a large-scale reactor

| Hₘᵢₙ Liquid | Hₘₐₓ Liquid | Washing | Washing |
|---|---|---|---|
| level (cm) | level (cm) | volume (l) | duration (min) |
| 3 | 4 | 25.9 | 11.3 |
| 6 | 7 | 39.4 | 15.8 |

### Example 5

The influence of the maximum liquid level above the resin on the piperidine washing efficiency.

The same stainless-steel reactor as in example 2 is used. Furthermore, an Fmoc-Ala-Gly-Rink Amide-MBHA-Resin (height 10 cm) is loaded into the reactor and homogenized with a DMF/piperidine solvent according to the protocol of example 2. The stirrer is stopped and the washing solvent, DMF, is introduced homogeneously so as not to disturb the resin bed and so that it remains horizontal on its surface. The discontinuous washing solvent introduction is carried out when the low-level liquid threshold h(min) is reached in order to limit the back-mixing phenomena and thus optimize the washing step. After solvent introduction, the liquid level reaches the maximum level h(max) depending on the inflow and the portion volume.

The resin bed is distributed homogeneously and horizontally in the reactor. The washing solvent (DMF) is introduced homogeneously on the surface of the liquid and the reactor wall to remove all traces of the agent to be removed. The solvent (DMF) is introduced at a flowrate of 600 l/h when the low-level liquid threshold h(min) of 3.0 cm is reached and discontinued at a high-level liquid threshold h(max) as indicated in table 6. The reactor is continuously drained at a superficial velocity of 212 cm/h. The liquid phase is discharged at a flowrate of 150 liter/hour. The percolation wash is performed until reaching the desired endpoint of 100 ppm of piperidine.

TABLE 6
Washing efficiency according to the maximum
liquid level in a large-scale reactor

| Hₘₐₓ Liquid level | Washing | Washing duration |
|---|---|---|
| (cm) | volume (l) | (min) |
| 4 | 25.9 | 11.3 |
| 6.3 | 28.8 | 12.3 |

### Example 6

Coupling solution washing efficiency as a function of the washing method.

The same stainless-steel reactor as in examples 2 to 5 is used. The reactor is loaded with a NH2-Gly-Rink Amide-MBHA-Resin (height 10 cm) and a coupling solution containing Fmoc-Ala-OH/DIC/Oxyma (3/3/3 eq) in DMF (0.5M) is added. The resin is homogenized by stirring until reaching the completion (reaction endpoint) of the reaction monitored by an offline analysis (i.e. Kaiser test). Kaiser test based on the reaction of ninhydrin with the primary amine from the N-terminal amine group of the deprotected peptide-resin. Subsequently the coupling solution is discharged from the reactor in case of batch washing.

The (displacement) endpoint is determined by UV after 5 batch washes (5.2 L each) and consequently percolation wash was performed until reaching the same endpoint (0.254 uA for an optical path of 0.5 mm at a wavelength of 301 nm).

Discontinuous percolation: The stirrer is stopped and the resin bed is distributed homogeneously and horizontally in the reactor. The washing solvent is introduced homogeneously on the surface of the liquid and the reactor wall to remove all traces of the agent (piperidine) to be removed. The solvent is introduced at 600 l/h when the low-level liquid threshold h(min) of 3.0 cm is reached and the introduction is stopped when the high-level liquid threshold h(max) of 4 cm is reached. The reactor is continuously drained during the percolation wash at a superficial speed of 212 cm/h (and a flow-rate of 150 l/h of the discharged liquid).

Batch wash: The washing solution (5.2 liters of DMF) is introduced homogeneously and stirred for 5 minutes. The reactor is then drained.

TABLE 6
Washing after coupling efficiency according to
the washing method in a large-scale reactor

| | Washing | Washing duration |
|---|---|---|
| Washing method | volume (l) | (min) |
| Batch wash | 25.9 | 25 |
| Discontinuous | 13.1 | 6.7 |
| percolation | | |

1. A process for displacing compounds comprised in the liquid phase and solid phase of reaction steps of a heterogeneous chemical reaction protocol comprising a liquid phase and a solid phase, the solid phase being present in particulate form dispersed in the liquid phase, for the formation of a target-compound synthesized by the consecutive introduction of sub-units (compounds) by repetitive (recurrent) cycles, each cycle comprising reaction steps, the process comprising providing:
a reactor where the reaction steps occur, the reactor comprising: a gas phase, a liquid phase, a solid phase and means for discharging the liquid phase from the reactor without essentially removing the solid phase from the reactor;
removing the liquid phase from the reactor; wherein a displacement liquid is discontinuously fed to the reactor, and wherein a layer of liquid phase is always maintained over the solid phase.

2. The process according to claim 1, wherein the liquid phase from the reactor is continuously removed.

3. The process according to claim 1, comprising determination of a gas-liquid phase boundary and a liquid-solid phase boundary thereby establishing a height (h) of the liquid phase over the solid phase, wherein the height (h) is never zero.

4. The process according to claim 3, wherein the height (h) is fluctuating over time.

5. The process according to claim 3, wherein the height (h) of the liquid phase is fluctuating up to about 1000%.

6. The process according to claim 3, wherein a minimum height h(min) of the liquid phase over the solid phase is about 0.5 cm.

7. The process according to claim 3, wherein a maximum height h(max) of the liquid phase over the solid phase is about 20 cm.

8. The process according to claim 3, wherein a difference between a maximum height h(max) of the liquid phase over the solid phase and a minimum height h(min) of the liquid phase over the solid phase is from about 0.1 cm up to about 20 cm.

9. The process according to claim 3, wherein a ratio between a minimum height h(min) of the liquid phase over the solid phase and a maximum height h(max) of the liquid phase over the solid phase is between about 1.1 and about 6 wherein the ratio is defined as h(max)/h(min).

10. The process according to claim 1, wherein a superficial velocity of the displacement liquid (over the solid phase) in the reactor is in the range of from about 100 cm/hour up to about 400 cm/hour.

11. The process according to claim 1, wherein a flowrate of the displacement liquid to the reactor is higher than a flow rate of liquid phase discharged from the reactor.

12. The process according to claim 11, wherein the flowrate of the displacement liquid to the reactor is from about 110% up to about 500% based on the flowrate of the liquid phase discharged from the reactor.

13. The process according to claim 1, wherein a gas-liquid phase boundary and a liquid-solid phase boundary are monitored by at least one detector.

14. The process according to claim 13, wherein the detector is selected from ultrasound, radar, or laser sensors.

15. The process according to claim 13, wherein the acquisition of data from the at least one detector is conducted when no displacement liquid is fed to the reactor.

16. The process according to claim 1, wherein a target product is selected from peptides and oligonucleotides and the sub-units are selected from amino acids, nucleosides, and derivatives thereof.

17. The process according to claim 1, wherein a target product is a peptide and the sub-units are amino acids and derivatives thereof.

18. The process according to claim 17, wherein the heterogeneous chemical reaction is solid phase peptide synthesis (SPPS).

19. The process according to claim 16, wherein the reactor comprises means for facilitating mass-transfer between the liquid phase and the solid phase and at least an inlet and an outlet.

20. The process according to claim 19, wherein the means for facilitating mass-transfer between the liquid phase and the solid phase is not active when the displacement liquid is drained from the reactor.

21. The process according to claim 19, wherein the means for facilitating mass-transfer between the liquid phase and the solid phase is switched off when the reaction step of a cycle has reached a predetermined reaction endpoint and remains switched off (inoperable) during a period (of time) defined by the first feed of the displacement liquid until a displacement endpoint is reached.

22. The process according to claim 19, wherein the means for facilitating mass-transfer between the liquid phase and the solid phase is switched on after an attainment of a displacement endpoint thereby providing a homogenized dispersion of solid phase in the liquid phase and establishing if the endpoint is maintained.

23. The process according to claim 16, wherein the solid phase is selected from silica containing materials and polymeric materials comprising reactive sites which are capable of covalently link the polymeric material with a sub-unit, optionally through a linker, of a target compound.

24. The process according to claim 1, wherein the liquid phase removed from the reactor is monitored by at least one detector for the monitoring of at least one species in the removed liquid phase to establish an endpoint.

25. A process for synthesizing peptides or oligonucleotides by an application of a solid phase peptide synthesis protocol comprising the process as defined by claim 1.

26. A process for displacing compounds comprised in the liquid phase and solid phase of reaction steps of a solid phase peptide synthesis reaction protocol for the formation of a target peptide synthesized by the consecutive introduction of amino acids and derivatives thereof by repetitive (recurrent) cycles, each cycle comprising reaction steps comprising a liquid phase and a solid phase, the solid phase being present in particulate form dispersed in the liquid phase, the process comprising providing:
a tank reactor where the reaction steps occur, the reactor comprising: an inlet, an outlet, means for facilitating mass-transfer between the liquid phase and the solid phase, a gas phase, liquid phase, a solid phase and means for discharging the liquid phase from the reactor without essentially removing the solid phase from the reactor;
removing the liquid phase from the reactor through the outlet; wherein a displacement liquid is discontinuously fed through the inlet to the reactor, and wherein a liquid phase is always maintained over the solid phase until a predetermined endpoint is reached.

27. The process according to claim 26, wherein the liquid phase is continuously removed from the reactor.

28. The process according to claim 26, further comprising the sedimentation of the solid phase thereby forming a liquid-solid phase boundary, a liquid phase over the solid phase and a gas-liquid phase boundary.

29. The process according to claim 28, wherein the reactor comprises at least one sensor capable of detecting the solid-liquid phase boundary and the gas-liquid phase boundary thereby providing the height h of the liquid phase over the solid phase.

30. The process according to claim 29, wherein a minimum height h(min) of the liquid phase over the solid phase is about 0.5 cm.

31. The process according to claim 29, wherein a maximum height h(max) of the liquid phase over the solid phase is about 20 cm.

32. The process according to claim 29, wherein a difference between a maximum height h(max) of the liquid phase over the solid phase and a minimum height h(min) of the liquid phase over the solid phase is from about 0.1 cm up to about 20 cm.

33. The process according to claim 26, wherein a superficial velocity of the displacement liquid (over the solid phase) in the reactor is in the range of from about 100 cm/hour up to about 400 cm/hour.

34. A process for displacing compounds (and/or solvents) comprised in a liquid phase and solid phase of reaction steps of a solid phase peptide synthesis, the process comprising providing a reactor where the reaction steps occur, the reactor comprising a liquid phase and a solid phase, means for discharging the liquid phase from the reactor without essentially removing the solid phase from the reactor, removing the liquid phase from the reactor while displacement liquid is discontinuously (intermittently) fed to the reactor, and wherein a continuous layer of liquid phase is maintained over the solid phase.
